SEQUENCE LISTING (17 sequences)

SEQ ID NO: 1 - DNA, 21 bp, Artificial
  Sequencing Primer
ggattagataccctggtagtc

SEQ ID NO: 2 - DNA, 19 bp, Artificial
  Sequencing Primer
cccgggaacgtattcaccg

SEQ ID NO: 3 - DNA, 550 bp, Bacteroides sp.
gcacagtaaacgatgaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcagcc

SEQ ID NO: 4 - DNA, 560 bp, Porphyromonas levii
cgctgtaaacgatgattactcagagtatgcgatataatgtatgctctcaagcgaaagcgttaagtaatccacctggggagtacgtcggcaacgatgaaactcaaaggaattgacgggggcccgcacaagcggaggaacatgtggtttaattcgatgatacgcgaggaaccttacctgggattgaaatgtatatgccggtatcccgaaaggggtgctattcacttcggtgacgtatatgtaggtgctgcatggttgtcgtcagctcgtgccgtgaggtgtcggcttaagtgccataacgagcgcaacccttatcgtcagttgctagcaggtaaagctgaggactctggcgagactgccgtcgtaaggcgagaggaaggtggggatgacgtcaaatcagcacggcccttatatccagggcgacacacgtgttacaatggtgaggacaaagggtcgctacccggtgacgggatgccaatctccaaacctcatctcagttcggatcggagtctgcaactcgactccgtgaagctggattcgctagtaatcgcgcatcagccatg

SEQ ID NO: 5 - DNA, 520 bp, Tannerella forsythensis
tactaggagtttgcgatatacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggattgaaatgtagacgacggacagtgagagctgtcttcccttcggggcgtctatgtaggtgctgcatggttgtcgtcagctcgtgccgtgaggtgtcggcttaagtgccataacgagcgcaaccctgactgtcagttgctaacaggttaagctgaggactctggcgggactgccggcgtaagctgtgaggaaggttgggatgacgtcaaatcagcacggcccttacatccggggcgacacacgtgttacaatggcagggacaaagggcagctaccgggcgaccggatgccaatctccaaaccctgtctcagttcggatcggagtctgcaactcgactccgtgaagctggattcgctag

SEQ ID NO: 6 - DNA, 1496 bp, Bacteroides sp.
aggcttacacatgcaagtcgaggggcagcattatcttagcttgctaagatagatggcgaccggcgcacgggtgagtaacacgtatccaaccttccggttactcggggataggctttcgaaagaaagattaatacccgatgttgcgtatctttctcctgaaagatacgccaaaggattccggtaaccgatggggatgcgttccattaggcagttggcggggtaacggcccaccaaaccttcgatggataggggttctgagaggaaggtcccccacattggaactgagacacggtccaaactcctacgggaggcagcagtgaggaatattggtcaatggacggaagtctgaaccagccaagtagcgtgaaggatgactgccctctgggttgtaaacttcttttatacgggaataacatgaggtacgcgtaccttattgcatgtaccgttatgaataagcatcggctaactccgtgccagcagccgcggtaatacggaggatgcgagcgttatccggatttattgggtttaaagggagcgtaggtgggatattaagtcagctgtgaaagtttggggctcaaccttaaaattgcagttgatactggtttccttgagtacggtacaggtgggcggaattcgtggtgtagcggtgaaatgcttagatatcacgaagaactccgatcgcgaaggcagctcaccgggccggaactgacactgatgctcgaaagtgcgggtatcaaacaggattagataccctggtagtccgcacagtaaacgatgaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcagccacggcgcggtgaatacgttcccgggccttgtacacaccgcccgtcaagccatgaaagccgggggtacctgaagtacgtaaccgcgaggatcgtcctagggtaaacctggtgattggggctaagtcgtaacaaggtagccgtaccggaaggtgcggctggaacacctcctttctggagcgatgcc

SEQ ID NO: 7 - DNA, 563 bp, Bacteroides sp.
cagtaaacgatgaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcagccacggcgcggtgaatac

SEQ ID NO: 8 - DNA, 563 bp, Bacteroides sp.; identical to SEQ ID NO: 7

SEQ ID NO: 9 - DNA, 565 bp, Bacteroides sp.
gcacagtaaacgatgaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcaaccacggcgcggtgaata

SEQ ID NO: 10 - DNA, 564 bp, Bacteroides sp.
acagtaaacgatgaaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcaaccacggcgcggtgaata

SEQ ID NO: 11 - DNA, 566 bp, Bacteroides sp.
  misc_feature (547)..(547): n is a, c, g, or t
cgcacagtaaacgatgaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatnaccacggngcggtgaatac

SEQ ID NO: 12 - DNA, 565 bp, Bacteroides sp.
gcacagtaaacgatgaatactcgctgtttgcgatacactgtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcagccacggcgcggtgaata

SEQ ID NO: 13 - DNA, 565 bp, Bacteroides sp.
cacagtaaacgatgaatactcgctgtttgcgatacacggtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcagccacggcgcggtgaatac

SEQ ID NO: 14 - DNA, 564 bp, Bacteroides sp.
acagtaaacgatgaatactcgctgtttgcgatacacggtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcgctggcttttaccggaaacggtattttcttcggaccagcgtgaaggtgctgcatggttgtcgtcagctcgtgccgtgaggtgtcggcttaagtgccataacgagcgcaacccttatctttagttactaacagttttgctgaggactctaaagagactgccgtcgtaagatgcgaggaaggtggggatgacgtcaaatcagcacggcccttacgtccggggctacacacgtgttacaatggggagcacagcaggttgctacacggcgacgtgatgccaatccgtaaaactcctctcagttcggatcgaagtctgcaacccgacttcgtgaagctggattcgctagtaatcgcgcatcagccacggcgcggtgaatac

SEQ ID NO: 15 - DNA, 565 bp, Bacteroides sp.; identical to SEQ ID NO: 13

SEQ ID NO: 16 - DNA, 20 bp, Artificial
  Sequencing Primer
gagtttgatcctggctcagg

SEQ ID NO: 17 - DNA, 19 bp, Artificial
  Sequencing Primer
cccgggaacgtattcaccg

(19) United States
(12) Patent Application Publication
(10) Pub. No.: US 2006/0257429 A1
(43) Pub. Date: Nov. 16, 2006

(54) Vaccine for periodontal disease

(76) Inventors: Kimberly J. Dreier, Oakdale, CT (US); John Morgan Hardham, Kalamazoo, MI (US); Rajendra Krishnan, Portage, MI (US); David Ross McGavin, Portage, MI (US)

Correspondence Address: PHARMACIA & UPJOHN, 7000 Portage Road, KZO-300-104, KALAMAZOO, MI, 49001, US

(21) Appl. No.: 10/568,999

(22) PCT Filed: Oct. 11, 2004
(86) PCT No.: PCT/IB04/03310

§ 371 Date: May 3, 2006

Related U.S. Application Data
(60) Provisional application No. 60/513,724, filed on Oct. 23, 2003.

(51) Int. Cl. A61K 39/02; C12Q 1/68; C12Q 1/04; C12N 1/20

(52) U.S. Cl. 424/234.1; 435/6; 435/34; 435/252.1

(57) ABSTRACT

The present invention relates to novel bacterial isolates identified by their 16S rRNA DNA, that cause periodontal disease in companion animals, and vaccines comprising such bacteria. Also provided are methods for treating and preventing periodontal disease and kits for detecting and treating periodontal disease kits for detecting and preventing periodontal disease.

### FIELD OF THE INVENTION

The present invention relates to novel bacterial isolates identified by their 16S rRNA DNA, that cause periodontal disease in companion animals, polynucleotide sequences contained therein, polypeptides encoded by such polynucleotide sequences and vaccines comprising such bacterial isolates that have been inactivated or attenuated, polynucleotides or polypeptides. Also provided are methods for treating and preventing periodontal disease and kits for detecting, treating, and preventing periodontal disease.

### BACKGROUND OF THE INVENTION

Periodontal disease comprises a group of infections involving supporting tissues of the teeth. These range in severity from mild and reversible inflammation of the gingiva (gum) to chronic destruction of periodontal-tissues (gingiva, periodontal ligament, and alveolar bone) with eventual exfoliation of teeth. The vast majority of experimental data concerning periodontal diseases is based on studies of humans or bacteria isolated from humans. Relatively little is known with respect to periodontal disease in non-human animals, such as companion animals, and in particular, dogs and cats.

From a microbiological standpoint, several features of this disease are of interest. The bacterial etiology is complex, with a variety of organisms responsible for the initiation and progression of disease in humans. Many, if not all, of these organisms may also be present in periodontally healthy individuals and can exist in commensal harmony with the host. It is known that in humans, successful colonizers of the teeth and subgingival area must coexist with many (over 600) other species of bacteria that inhabit these regions.

Both the calcified hard tissues of the tooth and the epithelial cells of the gingival are available for colonization. These tissues are exposed to host salivary secretions and gingival crevicular fluid (a serum exudate), both of which contain molecules that interact directly with bacteria and alter prevailing environmental conditions. The local environment imposes a variety of unique constraints upon the constituent microbiota of the supragingival tooth surface and the subgingival crevice (the channel between the tooth root and the gingiva that deepens into a periodontal pocket as disease progresses). Study of the pathogenesis of periodontal diseases in humans is complicated by the ecological intricacy of the microenvironment. However, it appears that disease episodes may ensue from a shift in the ecological balance between bacterial and host factors, as a result of, for example, alteration in the absolute or relative numbers of certain organisms, changes in pathogenic potential, or modulation of particular host factors.

The classification of the various manifestations of periodontal disease in humans is continually changing, and it will suffice to mention that diseases range in severity, rate of progression, and number of teeth affected and that different age groups can be susceptible following the eruption of primary teeth. The nature of the pathogenic agents varies among these disease entities, as well as among human patients and even between different disease sites within a patient. In general, however, severe forms of the disease are associated with a number of gram-negative anaerobic bacteria. Of this group, in humans, most evidence points to a pathogenic role for Porphyromonas (formerly Bacteroides) gingivalis. The presence of this organism, acting either alone or as a mixed infection with other bacteria, and possibly in concert with the absence of beneficial species and certain immunological responses in the host, appears to be essential for disease activity.

Initial entry of P. gingivalis into the human oral cavity is thought to occur by transmission from infected individuals. Other vectors would therefore also appear to be operational. These studies indicate that individuals are colonized by a single (or at least a predominant) genotype, regardless of site of colonization or clinical status. Strains of many different clonal origins, in contrast, are present in different individuals. This supports the concept that P. gingivalis is essentially an opportunistic pathogen, with virulence not being restricted to a particular clonal type.

In addition to P. gingivalis, Bacteroides spp. have also been associated with periodontitis in man. A novel Bacteroides species, Bacteroides forsythus, was originally isolated from anaerobic periodontal pockets (Tanner et al., “A study of the bacteria associated with advancing periodontitis in man”, Journal of Clinical Periodontology (1979), 6, 278-307). It was recently reclassified as Tannerella forsythensis based on various biochemical criteria (Sakamoto et al., “Reclassification of Bacteroides forsythus (Tanner et al. 1986) as Tannerella forsythensis corrig., gen. nov., comb. nov.”, International Journal of Systematic and Evolutionary Microbiology (2002), 52, 841-849).

While a great deal is known about periodontal disease in humans, very little is known about the same disease in companion animals. Although Porphyromonas species have also been implicated in disease in animals, these isolates have characteristics which distinguish them from their human counterparts (reviewed by Harvey in “Periodontal disease in dogs. Etiopathogenesis, prevalence, and significance”, Veterinary Clinics of North America—Small Animal Practice (1998), 28, 1111-1128). Fournier, D. et al. describe the isolation of an animal biotype of P. gingivalis from various animal hosts (“Porphorymonas gulae sp. nov., an Anaerobic, Gram-negative, Coccibacillus from the Gingival Sulcus of Various Animal Hosts”, International Journal of Systematic and Evolutionary Microbiology (2001), 51, 1179-1189). The authors hypothesize that this organism (P. gulae) represents a Porphyromonas species that is distinct from P. gingivalis. WO 03/054755 describes novel Porphyromonas isolates from dogs and cats, as well as methods and kits for treating and preventing periodontal disease.

Bacteroides species have also been isolated from subgingival sites in dogs diagnosed with periodontal disease (Forsblom et al., “Characterization of Anaerobic, Gram-Negative, Nonpigmented, Saccharolytic Rods from Subgingival Sites in Dogs”, Clinical Infectious Diseases (1997), 25, S100-106).

There remains a need for a safe and effective vaccine for treating and preventing periodontal disease in companion animals.

### SUMMARY OF THE INVENTION

The invention provides an isolated pigmented anaerobic bacterium which causes, either directly or in combination with other pathogenic agents, periodontal disease in companion animals.

In another embodiment, the present invention provides an isolated pigmented anaerobic bacterium or bacteria which causes, either directly or in combination with other pathogenic agents, periodontal disease in companion animals, wherein the bacterium or bacteria can be used to prepare a vaccine for treating or preventing periodontal disease in mammals including companion animals, wherein the vaccine comprises an immunologically effective amount of at least one bacteria or bacteria which has/have been inactivated or attenuated.

In one embodiment, the bacterium/bacteria is additionally selected from the group consisting of Bacteroides denticanoris, Porphyromonas levii, and Tannerella forsythensis.

Preferably, the bacterium/bacteria comprises a 16S rRNA DNA sequence at least about 95%, 95.5% 96%, 96.5%, 97%, 97.5% 98%, 98.5%, 99%, 99.5% homologous to a sequence selected from the group consisting of SEQ ID NOS: 3, 4, 5, 6, 7, 8, 9, 10, 11, 12, 13, 14 and 15

In yet a further embodiment, the present invention provides an isolated polynucleotide molecule comprising any of the nucleotide sequences selected from the group consisting of SEQ ID NOS: 3, 4, 5, 6, 7, 8, 9, 10, 11, 12, 13, 14 and 15 and homologues having at least about 95%, 95.5% 96%, 96.5%, 97%, 97.5% 98%, 98.5%, 99%, 99.5% homology thereto. The isolated polynucleotides of the invention include fragments and variants as defined below.

In another aspect, the present invention provides an immunogenic composition comprising at least one pigmented anaerobic bacteria according to the present invention, and a pharmaceutically acceptable carrier. The bacteria of the immunogenic composition may be live or inactivated. Optionally the immunogenic composition may include an adjuvant.

In a further aspect, the present invention provides a vaccine for treating or preventing periodontal disease in mammals including companion animals comprising an immunologically effective amount of at least one pigmented anaerobic bacteria according to the present invention, and a pharmaceutically acceptable carrier. The bacteria of the vaccine may be live or inactivated. Optionally the vaccine may include an adjuvant.

In another aspect the present invention provides a method for treating or preventing periodontal disease in mammals including companion animals comprising administering to a mammal in need thereof, a vaccine composition according to the present invention.

In another aspect the present invention provides a method for diagnosing periodontal disease in mammals including companion animals by analyzing a sample for bacteria, polypeptides or polynucleotides of the present invention, wherein the presence of the bacteria, polypeptides, or polynucleotides are indicative of disease. Preferably, the analyzing step includes analyzing the sample using a method selected from the group consisting of PCR, hybridization, and antibody detection.

In yet another aspect, the present invention provides a kit comprising, in at least one container, a composition for treating and preventing periodontal disease in mammals including companion animals comprising an effective amount of at least one Inactivated or attenuated isolated pigmented anaerobic bacteria, or a polypeptide, or polynucleotides derived from the pigmented anaerobic bacteria and a pharmaceutically acceptable carrier; The kit further comprises a set of printed instructions indicating that the kit is useful for treating or preventing periodontal disease in mammals. The kit may further comprise a means for dispensing said composition.

In still another aspect, the present invention provides a kit comprising in at least one container an isolated DNA molecule comprising a nucleotide sequence of at least about 15 contiguous nucleotides selected from any of SEQ ID NOS: 3, 4, 5, 6, 7, 8, 9, 10, 11, 12, 13, 14 and 15 which hybridizes under highly stringent conditions to the complement of any of the nucleotide sequences depicted in SEQ ID NOS: 3, 4, 5, 6, 7, 8, 9, 10, 11, 12, 13,14 and 15 and a second isolated DNA molecule comprising in a second container an isolated DNA molecule comprising a nucleotide sequence of at least about 15 contiguous nucleotides selected from the complement of any of the nucleotide sequences depicted in SEQ ID NOS: 3, 4, 5, 6, 7, 8, 9, 10, 11, 12, 13, 14 and 15 which hybridizes under highly stringent conditions to any of the nucleotide sequences depicted in SEQ ID NOS: 3, 4, 5, 6, 7, 8, 9, 10,11, 12, 13, 14 and 15 wherein the kit further comprises a set of instructions indicating that the kit is useful for the detection of Bacteroides, Porphyromonas, and Tannerella spp. Such a method may be used generally in all mammals including companion animals.

In a further aspect, the present invention provides a hybridization kit comprising in at least one container an isolated DNA molecule comprising a nucleotide sequence of at least about 15 contiguous nucleotides selected from any of SEQ ID NOS: 3, 4, 5, 6, 7, 8, 9, 10, 11, 12, 13, 14 and 15 or its complement, wherein the hybridization is specific to Bacteroides, Porphyromonas, and Tannerella spp. and wherein the kit further comprises a set of instructions indicating that the kit Is useful for the detection of Bacteroides, Porphyromonas, and Tannerella spp. Preferably, the hybridization is performed under highly stringent conditions.

The invention further provides a biologically pure culture of bacteria, wherein the bacteria comprise a 16S rRNA DNA sequence at least about 95%, 95.5% 96%, 96.5%, 97%, 97.5% 98%, 98.5%, 99%, 99.5% homologous to a sequence selected from the group consisting of SEQ ID NOS: 3, 4, 5, 6, 7, 8, 9, 10, 11, 12, 13, 14 and 15.

The invention also provides a biologically pure culture of bacteria which is ATCC PTA-5881 or a culture having all of the identifying characteristics of ATCC PTA-5881. The invention also provides a biologically pure culture of bacteria which is ATCC PTA-5882 or a culture having all of the identifying characteristics of ATCC PTA-5882. The invention provides a biologically pure culture of bacteria which is ATCC PTA-6063 or a culture having all of the identifying characteristics of ATCC PTA-6063.

The invention also comprises isolated polynucleotides and polypeptides derived from the bacteria of the invention which have utility as a vaccine for treating or preventing periodontal disease in mammals including companion animals.

### BRIEF DESCRIPTION OF THE FIGURES

FIG. 1. Canine and feline BPAB isolate characterization

FIG. 2. The results of RapID ANA II testing for B. denticanoris B78^{T} as well as six control bacteria.

FIG. 3. Neighbor-joining phylogenetic tree for representatives from the Bacteroidetes class. The phylogenetic tree was generated using the CLUSTAL X version 1.81 and NJ Plot software programs (both available from ftp://ftp-igbmc.u-strasbg.fr/pub/ClustalX/). The tree was rooted to the Escherichia coli 16S rRNA gene sequence (accession number J01695) (data not shown). Bootstrap analysis was performed using 1000 replicates. Bootstrap values are presented graphically (•>950; ▪, >850; o, >700; □, >500; no designation, <500). The scale bar represents 0.01 substitutions per nucleotide position. The arrow indicates the location of B. denticanoris B78^{T}. Accession numbers: P. gingivalis ATCC 33277, J01695; P. gulae B243, AF285874; P. cansulci VPB 4875, X76260; P. salivosa NCTC 11632, L26103; P. endodontalis ATCC 35406, AY253728; T. forsythensis ATCC 43037, AB035460; Bacteroides cf. forsythus oral clone BU45, AF385565; B. merdae ATCC 43184T, X83954; B. distasonis ATCC 8503, M86695; Equine fecal bacterium 118ds10, AY212569; D. shahii strain CCUG 43457, AJ319867; A. putredinis ATCC 29800, L16497; R. microfusus ATCC 29728, L16498; Swine fecal bacterium FPC111, AF445205; Bacteroides sp. 139, AF319778; B. fragilis ATCC 25285T, X83935; B. thetaiotaomicron strain 17.4, AY319392; B. acidofaciens strain A37, AB021163; B. denticanoris B7₈^{T}, AY549431; Bacteroides sp. 0103-800, AJ416906; Uncultured Bacteroidetes Bisii27, UBA318179; P. bivia ATCC 29303. L16475; P. nigrescens ATCC 25261, L16479; P. intermedia ATCC 25611, L16468; P. denticola ATCC 35308, L16467; and P. buccae ATCC 33690, L16478.

FIG. 4. Neighbor-joining phylogenetic tree for clinical isolates of B. denticanoris. The phylogenetic tree was generated as in FIG. 1. The tree was rooted to the Bacteroides sp. 0103-800 16S rRNA gene sequence (accession number AJ416906). The scale bar represents 0.001 substitutions per nucleotide position. Only one member from each 16S rRNA sequence cluster is shown. Accession numbers: B. denticanoris B7₈^{T}, AY549431; B. denticanoris B80, AY549432; B. denticanoris B83, AY549433; B. denticanoris B241, AY549434; B. denticanoris B242, AY549435; B. denticanoris B342, AY549436; B. denticanoris B458, AY549437; B. denticanoris B473, AY549438; B. denticanoris B474, AY549439; and B. denticanoris B476, AY549440.

FIG. 5. Pathogenicity testing of B. denticanoris B78^{T} in the oral mouse model of periodontal disease. Sixteen mice were used for each test group. Mice were treated as described. Forty-two days post challenge, the mice were sacrificed, the jaws defleshed and stained. Fourteen independent measurements of the CEJ-ABC distance were taken on each jaw. The average CEJ-ABC measurement for each group is shown. The standard error for each group is indicated. The statistical significance between the two groups is shown.

### BRIEF DESCRIPTION OF THE SEQUENCE LISTING

Seq ID No. 1—Sequencing Primer
Seq ID No. 2—Sequencing Primer
Seq ID No. 3—DNA encoding a portion of the 16S rRNA from Bacteroides denticanoris (B78)
Seq ID No. 4—DNA encoding a portion of the 16S rRNA from Porphyromonas levii (B222)
Seq ID No. 5—DNA encoding a portion of the 16S rRNA from Tannerella forsythensis (B343-24)
Seq ID No. 6—DNA encoding a portion of the 16S rRNA from Bacteroides denticanoris (B78) (full length)
Seq ID No. 7—DNA encoding a portion of the 16S rRNA from Bacteroides denticanoris (B80)
Seq ID No. 8—DNA encoding a portion of the 16S rRNA from Bacteroides denticanoris (B83)
Seq ID No. 9—DNA encoding a portion of the 16S rRNA from Bacteroides denticanoris (B241)
Seq ID No. 10—DNA encoding a portion of the 16S rRNA from Bacteroides denticanoris (B242)
Seq ID No. 11—DNA encoding a portion of the 16S rRNA from Bacteroides denticanoris (B342)
Seq ID No. 12—DNA encoding a portion of the 16S rRNA from Bacteroides denticanoris (B458)
Seq ID No. 13—DNA encoding a portion of the 16S rRNA from Bacteroides denticanoris (B473)
Seq ID No. 14—DNA encoding a portion of the 16S rRNA from Bacteroides denticanoris (B474)
Seq ID No. 15—DNA encoding a portion of the 16S rRNA from Bacteroides denticanoris (B476)
Seq ID No. 16—Sequencing Primer
Seq ID No. 17—Sequencing Primer

### DETAILED DESCRIPTION OF THE INVENTION

### Bacterial Isolates

The present invention provides isolated anaerobic bacteria, identified by their 16S rRNA DNA sequences, which can cause periodontal disease and various other diseases and clinical manifestations in companion animals. More specifically, the bacteria are selected from the genera Bacteroides, Porphyromonas, and Tannerella.

In addition the invention provides a novel, anaerobic bacteria/bacterium causing periodontal disease in companion animals. The novel isolate induces alveolar bone loss in a mouse model of experimental periodontal disease The cellular morphology and biochemical properties of the bacterial isolate indicates that it is a member of the genus Bacteroides. Comparison of the 16S rRNA gene sequence suggested that the bacteria represented a previously undefined species within the genus Bacteroides based on biochemical, molecular phylogenetic, and pathogenic evidence, which we have designated Bacteroides denticanoris sp. nov. The type strain of Bacteroides denticanoris is strain B78^{T} (=ATCC PTA-5881). Preferably, therefore the isolated bacteria of the present invention include Bacteroides denticanoris (B78), Porphyromonas levii (B222), and Tannerella forsythensis (B343-24), although other species or strains are encompassed by the invention. In a preferred embodiment, the isolated bacteria of the present invention can be identified by their 16S rRNA DNA sequences shown in SEQ ID Nos. 3, 4, 5, 6, 7, 8, 9, 10, 11, 12, 13, 14 and 15.

The diseases caused by infection with the bacteria of the present invention include, but are not limited to, companion animal periodontal disease, companion animal oral malodor (halitosis), bovine foot rot, canine coronary heart disease and canine systemic infections. Bacteria within these genera have also been connected with various human diseases, including coronary heart disease, parotitis, oral malodor, gingivitis, periodontis, stroke, atherosclerosis, hyperlipidemia, bacterial vaginosis, intrauterine growth retardation (IUGR), and increased incidence of pre-term delivery of low birth weight infants.

The present invention provides isolated polynucleotide molecules of bacterial species. The present invention also provides polynucleotide sequences having at least about 90% homology, preferably at least about 95%, 95.5%. 96%, 96.5%, 97%, 97.5%, 98%, 98.5%, 99%, sequence identity to any of SEQ 3, 4, 5, 6, 7, 8, 9, 10, 11, 12, 13, 14 and 15.

In addition, the present invention provides polynucleotide sequences that hybridize under stringent conditions to the complement of any of the polynucleotide sequences shown in SEQ ID 3, 4, 5, 6, 7, 8, 9, 10, 11, 12, 13, 14 and 15.

In another specific embodiment, a nucleic acid which is hybridizable to any of the polynucleotide sequences depicted in SEQ ID 3, 4, 5, 6, 7, 8, 9, 10, 11, 12, 13, 14 and 15, or their complements, under conditions of high stringency is provided. By way of example and not limitation, procedures using such conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/mL denatured salmon sperm DNA. Filters are hybridized for 48 h at 65° C. in prehybridization mixture containing 100 μg/mL denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography.

Other conditions of high stringency which may be used depend on the nature of the nucleic acid (e.g. length, GC content, etc.) and the purpose of the hybridization (detection, amplification, etc.) and are well known in the art. For example, stringent hybridization of an oligonucleotide of approximately 15-40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C.

In a preferred embodiment, after hybridization, wash conditions are as follows. Each membrane is washed two times each for 30 minutes each at 45° C. in 40 mM sodium phosphate, pH 7.2, 5% SDS, 1 mM EDTA, 0.5% bovine serum albumin, followed by four washes each for 30 minutes in sodium phosphate, pH 7.2, 1% SDS, 1 mM EDTA. For high stringency hybridization, the membranes are additionally subjected to four washes each for 30 minutes in 40 mM sodium phosphate, pH 7.2, 1% SDS, 1 mM EDTA at 55° C., followed by four washes each for 30 minutes in sodium phosphate, pH 7.2, 1% SDS, 1 mM EDTA at 65° C.

The present invention further provides vaccines and vaccine formulations which, when administered to a companion animal in a therapeutically effective amount, are useful in treating or preventing (i.e., conferring resistance) to periodontal disease in a companion animal.

In one embodiment, the present invention provides a vaccine that comprises at least one attenuated (modified live) or inactivated whole cell preparation (bacterin). In another embodiment, the vaccine comprises a subunit fraction from one or more bacterial species,capable of inducing an immune response.

The attenuated (modified live) or inactivated vaccines (bacterins) can be present in combination with other known vaccine formulation components such as with compatible adjuvants, diluents, or carriers.

### DEFINITIONS AND ABBREVIATIONS

The term “identity” or “percentage of sequence identity” for nucleotide sequences is determined by comparing two optimally aligned sequences over a comparison window, wherein optimal alignment provides the highest order match and can introduce nucleotide additions or to the test or reference sequence. The percentage identity is determined by calculating the percentage of nucleotides that are identical between the test and reference sequence at each position over the entire sequence. Optimal sequence alignment and percentage identity can be determined manually, or more preferably by a computer algorithm including but not limited to TBLASTN, FASTA, GAP, BESTFIT, and CLUSTALW (Altschul et al., 1990, J. Mol. Biol. 215(3):403-10; Pearson and Lipman, 1988, Proc. Natl. Acad. Sci. USA 85(8):2444-8; Thompson, et al., 1994, Nucleic Acids Res. 22(22):4673-80; Devereux et al., 1984, Nuc. Acids. Res. 12:387-395; Higgins, et al., 1996, Methods Enzymol. 266:383-402). Preferably, the NCBI Blast Server (http://www.ncbi.nlm.nih.gov) set at the default parameters is used to search multiple databases for homologous sequences.

The term “heterologous”, when used herein means derived from a different bacterial species or strain.

The term “homology”, “homologous”, and the like, when used herein means the degree of identity shared between polynucleotide or polypeptide sequences.

The term “homologous”, when used in reference to a bacterial species means the same bacterial species or strain.

The term “isolated” when used herein means removed from its naturally occurring environment, either alone or in a heterologous host cell, or chromosome or vector (e.g., plasmid, phage, etc.).

The terms “isolated anaerobic bacteria”, “isolated bacteria”, “isolated bacterial strain” and the like refer to a composition in which the bacteria are substantially free of other microorganisms, e.g., in a culture, such as when separated from it naturally occurring environment. The term “biologically pure culture” when applied to the bacteria of the invention refers to a culture of bacteria substantially free of other microorganisms.

The term “isolated polynucleotide” indicates a composition in which the isolated nucleotide comprises at least 50% of the composition by weight. More preferably, the isolated polynucleotide comprises about 95%, and most preferably 99% by weight of the composition.

The term “functionally equivalent” as utilized herein, refers to a recombinant polypeptide capable of being recognized by an antibody specific to native polypeptide produced by the bacteria which causes periodontal disease in companion animals, or a recombinant polypeptide capable of eliciting or causing a substantially similar immunological response as that of the native protein from the endogenous bacteria. Thus, an antibody raised against a functionally equivalent polypeptide also recognizes the native polypeptide produced by the bacteria which causes periodontal disease in companion animals.

The term “immunogenicity” refers to the capability of a protein or polypeptide to elicit an immune response directed specifically against the bacteria that causes periodontal disease in companion animals.

The term “antigenicity” refers to the capability of a protein or polypeptide to be immunospecifically bound by an antibody raised against the protein or polypeptide.

The term “antibody”, as used herein, refers to an immunoglobulin molecule able to bind to an antigen. Antibodies can be a polyclonal mixture or monoclonal. Antibodies can be intact immunoglobulins derived from natural sources or from recombinant sources, or can be immunoreactive portions of intact immunoglobulins. Antibodies can exist in a variety of forms including, for example, as, Fv, Fab′, F(ab′)₂, as well as in single chains.

The term “companion animal”, as used herein, refers to any non-human animal in captivity considered to be a pet. These may include, but are not restricted to, dogs, cats, horses, rabbits, monkeys, and rodents, including mice, rats, hamsters, gerbils, and ferrets.

The term “protection”, “protecting”, and the like, as used herein with respect to a vaccine, means that the vaccine prevents or reduces the symptoms of the disease caused by the organism from which the antigen(s) used in the vaccine is derived. The terms “protection” and “protecting” and the like, also mean that the vaccine can be used to “treat” the disease or one of more symptoms of the disease that already exists in a subject.

The term “therapeutically effective amount” refers to an amount of the bacteria, or a subunit, (e.g., polypeptides, polynucleotide sequences) and combinations thereof sufficient to elicit an immune response in the subject to which it is administered. The immune response can comprise, without limitation, induction of cellular and/or humoral immunity.

The term “preventing infection” means to prevent or inhibit the replication of the bacteria which cause periodontal disease in companion animals, to inhibit transmission of the bacteria, or to prevent the bacteria from establishing itself in its host, or to alleviate the symptoms of the disease caused by infection. The treatment is considered therapeutic if there is a reduction in bacterial load.

The term “pharmaceutically acceptable carrier” refers to a carrier medium that does not interfere with the effectiveness of the biological activity of the active ingredient and is not toxic to the subject to whom it is administered.

The term “therapeutic agent” refers to any molecule, compound or treatment, preferably an antibacterial, that assists in the treatment of a bacterial infection or a disease or condition caused thereby.

The term “fragment or variant thereof” refers to partial nucleotide sequences according to the present invention. Analogs are encompassed by the term “fragment or variant thereof”. Mutant polynucleotides which may possess one or more mutations which are deletions, insertions or substitutions of nucleotide residues are encompassed by the term “fragment or variant thereof”. Allelic variants are encompassed by the term “fragment or variant thereof”.

### Isolation and Characterization of Bacterial Species

Bacteria provided by the present invention can be obtained using known sampling, culture and isolation techniques. For example, microbial samples can be obtained from a population of companion animals, such as from dogs and cats, exhibiting periodontal disease. Evidence of periodontal disease can be observed using known measures, such as dogs with periodontal pockets >3 mm and cats with periodontal pockets >2 mm. Known parameters for characterizing periodontal disease such as dental indices (gingival index and periodontal index) and periodontal pocket depths can determined for the sample population of companion animals. Individual samples can be obtained from the periodontal pocket of a particular animal, maintained under anaerobic conditions and cultured using various known culture media.

Clinical isolates can be characterized using known techniques such as a number of biochemical tests, and 16S rRNA DNA sequence analysis to determine their genus and species. Individual isolates can be transferred to plates and antibiotic disks (Anaerobe Systems) can be placed on the agar surface to determine the antibiotic resistance patterns of each isolate. Purified colonies can also be subjected to known indole and catalase tests (Anaerobe Systems). Lipase and lecithinase production patterns can be determined for individual isolates.

The isolates can be typed based on their 16S rRNA DNA sequence. Individual, well-isolated colonies can be utilized as a template for polymerase chain reactions (PCR) amplification of the 16S rRNA region using, for example, primers D0056 and D0057 (Seq. ID NO. 1 and Seq. ID NO. 2; Table 1). Optionally the full length 16S RNA can be amplified using, for example, the primers disclosed as Seq ID NO. 16 and 17.)

The resulting PCR products can be purified using available PCR preps kits (Promega Corp.; Madison, Wisc.) and pooled by isolate. The purified PCR products can then be desalted and subjected to DNA sequence analysis. The resulting DNA sequences can be used to search available DNA databases. The bacterial isolates can then be typed based on the closest match identified by database searches.

TABLE 1

DNA sequence identification listing. All oligonucleotide
primers were synthesized by Gibco-BRL (USA).

| | | | |
|---|---|---|---|
| SEQ ID NO. | Name | Target | DNA Sequence |

| | | | |
|---|---|---|---|
| 1 | D0056 | 16S rRNA | GGATTAGATACCCTGGTAGTC |
| 2 | D0057 | 16S rRNA | CCCGGGAACGTATTCACCG |
| 3 | Bacteroides | (Not | GCACAGTAAACGATGAATACTCGCTGTTT |
| | denticanoris | applicable) | GCGATACACTGTAAGCGGCCAAGCGAAA |
| | (B78) 16S rRNA | | GCGTTAAGTATTCCACCTGGGGA |
| | polynucleotide | | GTACGCCGGCAACGGTGAAACTCAAAGG |
| | sequence | | AATTGACGGGGGCCCGCACAAGCGGAG |
| | | | GAACATGTGGTTTAATTCGATGATA |
| | | | CGCGAGGAACCTTACCCGGGCTTAAATT |
| | | | GCGCTGGCTTTTACCGGAAACGGTATTT |
| | | | TCTTCGGACCAGCGTGAAGGTGCT |
| | | | GCATGGTTGTCGTCAGCTCGTGCCGTGA |
| | | | GGTGTCGGCTTAAGTGCCATAACGAGCG |
| | | | CAACCCTTATCTTTAGTTACTAAC |
| | | | AGTTTTGCTGAGGACTCTAAAGAGACTG |
| | | | CCGTCGTAAGATGCGAGGAAGGTGGGG |
| | | | ATGACGTCAAATCAGCACGGCCCTT |
| | | | ACGTCCGGGGCTACACACGTGTTACAAT |
| | | | GGGGAGCACAGCAGGTTGCTACACGGC |
| | | | GACGTGATGCCAATCCGTAAAACTC |
| | | | CTCTCAGTTCGGATCGAAGTCTGCAACC |
| | | | CGACTTCGTGAAGCTGGATTCGCTAGTA |
| | | | ATCGCGCATCAGCC |
| 4 | Porphyromonas | (Not | CGCTGTAAACGATGATTACTCAGAGTATG |
| | levii (B222) | applicable) | CGATATAATGTATGCTCTCAAGCGAAAGC |
| | 16S rRNA | | GTTAAGTAATCCACCTGGGGAG |
| | polynucleotide | | TACGTCGGCAACGATGAAACTCAAAGGA |
| | sequence | | ATTGACGGGGGCCCGCACAAGCGGAGG |
| | | | AACATGTGGTTTAATTCGATGATAC |
| | | | GCGAGGAACCTTACCTGGGATTGAAATG |
| | | | TATATGCCGGTATCCCGAAAGGGGTGCT |
| | | | ATTCACTTCGGTGACGTATATGTA |
| | | | GGTGCTGCATGGTTGTCGTCAGCTCGTG |
| | | | CCGTGAGGTGTCGGCTTAAGTGCCATAA |
| | | | CGAGCGCAACCCTTATCGTCAGTT |
| | | | GCTAGCAGGTAAAGCTGAGGACTCTGGC |
| | | | GAGACTGCCGTCGTAAGGCGAGAGGAA |
| | | | GGTGGGGATGACGTCAAATCAGCAC |
| | | | GGCCCTTATATCCAGGGCGACACACGTG |
| | | | TTACAATGGTGAGGACAAAGGGTCGCTA |
| | | | CCCGGTGACGGGATGCCAATCTCC |
| | | | AAACCTCATCTCAGTTCGGATCGGAGTC |
| | | | TGCAACTCGACTCCGTGAAGCTGGATTC |
| | | | GCTAGTAATCGCGCATCAGCCATG |
| 5 | Tannerella | (Not | TACTAGGAGTTTGCGATATACAGTAAGCT |
| | forsythensis | applicable) | CTACAGCGAAAGCGTTAAGTAATCCACC |
| | (B343-24) 16S | | TGGGGAGTACGCCGGCAACGGTG |
| | rRNA | | AAACTCAAAGGAATTGACGGGGGCCCGC |
| | polynucleotide | | ACAAGCGGAGGAACATGTGGTTTAATTC |
| | sequence | | GATGATACGCGAGGAACCTTACCC |
| | | | GGGATTGAAATGTAGACGACGGACAGTG |
| | | | AGAGCTGTCTTCCCTTCGGGGCGTCTAT |
| | | | GTAGGTGCTGCATGGTTGTCGTCA |
| | | | GCTCGTGCCGTGAGGTGTCGGCTTAAGT |
| | | | GCCATAACGAGCGCAACCCTGACTGTCA |
| | | | GTTGCTAACAGGTTAAGCTGAGGA |
| | | | CTCTGGCGGGACTGCCGGCGTAAGCTG |
| | | | TGAGGAAGGTTGGGATGACGTCAAATCA |
| | | | GCACGGCCCTTACATCCGGGGCGAC |
| | | | ACACGTGTTACAATGGCAGGGACAAAGG |
| | | | GCAGCTACCGGGCGACCGGATGCCAAT |
| | | | CTCCAAACCCTGTCTCAGTTCGGAT |
| | | | CGGAGTCTGCAACTCGACTCCGTGAAGC |
| | | | TGGATTCGCTAG |

The following companion animal periodontal isolates were deposited with the American Type Culture Collection (ATCC), 10801 University Blvd., Manassas, Va., 20110, USA: Bacteroides denticanoris (B78; (PTA-5881), Porphyromonas levii (B222; (PTA-5882), and Tannerella forsythensis (B343-24; (PTA-6063).

### Cloning of Bacterial Nucleotide Sequences

There are several known methods or techniques that can be used to clone the nucleotide sequences of the present invention. For example, the sequences can be isolated as restriction fragments and cloned into cloning and/or expression vectors, the sequences can be PCR amplified and cloned into cloning and/or expression vectors, or the sequences can be cloned by a combination of these two methods.

Standard molecular biology techniques known in the art and not specifically described can be generally followed as described in Sambrook et al., Molecular Cloning: A Laboratory Manual, Cold Spring Harbor Laboratory Press, New York (1989); Ausubel et al., Current Protocols in Molecular Biology, John Wiley and Sons, Baltimore, Md. (1989); Perbal, A Practical Guide to Molecular Cloning, John Wiley & Sons, New York (1988); Watson et al., Recombinant DNA, Scientific American Books, New York; Birren et al (eds) Genome Analysis: A Laboratory Manual Series, Vols. 1-4 Cold Spring Harbor Laboratory Press, New York (1998); and methodology set forth in U.S. Pat. Nos. 4,666,828; 4,683,202; 4,801,531; 5,192,659 and 5,272,057. Polymerase chain reaction (PCR) is carried out generally as described in PCR Protocols: A Guide To Methods And Applications, Academic Press, San Diego, Calif. (1990).

Examples of methods useful in cloning and sequencing the polynucleotides of the present invention are provided in the Example.

### Antibody Production

Antibodies may either be monoclonal, polyclonal, or recombinant. Conveniently, the antibodies may be prepared against the immunogen or portion thereof, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W.H. Freeman and Co., 1992. Antibody fragments may also be prepared from the antibodies and include Fab, F(ab′)₂, and Fv by methods known to those skilled in the art.

In the production of antibodies, screening for the desired antibody can be accomplished by standard methods in immunology known in the art. Techniques not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980). In general, ELISAs and Western blotting are the preferred types of immunoassays. Both assays are well known to those skilled in the art. Both polyclonal and monoclonal antibodies can be used in the assays. The antibody can be bound to a solid support substrate or conjugated with a detectable moiety or be both bound and conjugated as is well known in the art (for a general discussion of conjugation of fluorescent or enzymatic moieties see Johnstone & Thorpe, Immunochemistry in Practice, Blackwell Scientific Publications, Oxford, 1982.) The binding of antibodies to a solid support substrate is also well known in the art (see for a general discussion, Harlow & Lane Antibodies: A Laboratory Manual, Cold Spring Harbor Laboratory Publications, New York, 1988 and Borrebaeck, Antibody Engineering—A Practical Guide, W.H. Freeman and Co., 1992). The detectable moieties contemplated for use in the present invention can include, but are not limited to, fluorescent, metallic, enzymatic and radioactive markers such as biotin, gold, ferritin, alkaline phosphatase, b-galactosidase, peroxidase, urease, fluorescein, rhodamine, tritium, ¹⁴C and iodination.

Where appropriate, other immunoassays such as radioimmunoassays (RIA) can be used as known in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521, as well as Sambrook et al, Molecular Cloning: A Laboratory Manual, Cold Springs Harbor, N.Y., 1989.

### Detection, Diagnostic, and Prevention Kits

The present invention further provides kits for the detection of Bacteroides, Porphyromonas, and Tannerella species. The kit includes reagents for analyzing a sample for the presence of said organisms, polypeptides, or nucleotide sequences of the present invention, wherein the presence of the nucleotide sequence is indicative of the presence of the organism. This method is valuable because disease can be diagnosed prior to the existence of symptoms and can therefore prevent the onset of the disease prior to the occurrence of damage to the patient. The presence of bacteria, polypeptides or nucleotide sequences can be determined using antibodies, PCR, hybridization, and other detection methods known to those of skill in the art.

In one embodiment, the kit provides reagents for the detection of antibodies against Bacteroides, Porphyromonas, or Tannerella spp. In certain embodiments, the kit can include a set of printed instructions or a label indicating that the kit is useful for the detection of Bacteroides, Porphyromonas, or Tannerella spp. In another embodiment, the kit provides reagents for the detection of Bacteroides, Porphyromonas, or Tannerella spp. nucleic acids. In one embodiment, the kit provides reagents for the PCR detection of Bacteroides, Porphyromonas, or Tannerella spp. nucleic acids and comprises in at least one container a first isolated DNA molecule comprising a fragment of at least about 15, 20, 25 or 30 nucleotides, which fragment hybridizes under stringent conditions to a DNA molecule comprising a sequence of at least 15, 30, 45, 60, 75, or 90 contiguous nucleotides, of any of the polynucleotides of SEQ ID NO:3-5, and a second isolated DNA molecule comprising a fragment of at least 15, 20,25, or 30 nucleotides, which fragment hybridizes under stringent conditions to a DNA molecule complementary to a DNA molecule having a sequence of at least 15, 30, 45, 60, 75, or 90 contiguous nucleotides of any of the polynucleotides of SEQ ID NO:3-5, which first and second DNA molecules can be used to specifically amplify a Bacteroides, Porphyromonas, or Tannerella spp. nucleic acid encoding a 16S rRNA which 16S rRNA is encoded by a DNA molecule selected from the group consisting of SEQ ID NOS: 3-5.

### Vaccine Formulation and Method of Administration

The vaccine of the present invention can be administered to a companion animal in an effective amount such that the vaccine therapeutically treats or confers resistance to or prevents periodontal disease in the companion animal. The vaccine of the present invention is useful in the control of bacteria that cause periodontal disease. The vaccines of the present invention can, in particular, be used in the field of veterinary medicine to treat companion animals and for the maintenance of public health against those bacteria described herein which are known to cause periodontal disease.

The vaccines of the present invention are of value In the control of bacteria that are injurious to, or spread or act as vectors of disease in man and companion animals, for example those described herein. The vaccines of the present invention are particularly useful in controlling bacteria that are present in companion animals for which purpose they can be administered using any known methods of administration, including, but not limited to, oral, parenteral, intranasal, subcutaneous, or topical.

According to a further aspect of the present invention, there is provided a composition comprising a vaccine of the present invention, in admixture with a compatible adjuvant, diluent or carrier. In a preferred embodiment, the vaccine formulation of the present invention is composed of an aqueous suspension or solution containing at least one bacteria of the present invention and/or at least one subunit protein, preferably buffered at physiological pH, in a form ready for injection.

The present invention further provides a method of treating or preventing a bacterial infection, which comprises treatment with an effective amount of a vaccine or vaccine formulation of the present invention. It is to be appreciated that reference to treatment includes prophylaxis as well as the alleviation of established symptoms of a bacterial infection.

The vaccines and vaccine formulations of the present Invention can be used to induce a response that prevents the pathological changes characteristic of periodontal disease caused by periodontal disease-causing bacteria. In a vaccine formulation, an immunogenic amount of the bacteria, purified protein, nucleic acid, or combinations thereof is desirably mixed with a suitable conventional vaccine adjuvants and physiologic vehicles, for use in mammals.

A vaccine formulation for preventing periodontal disease in companion animals can be produced using at least one of the isolated and purified inactivated or attenuated bacteria, purified polypeptides (such as native proteins, subunit proteins, or polypeptides) and admixing one or more or these with a compatible adjuvant, diluent, or carrier.

The present invention further provides for combination vaccines having at least one of the inactivated or attenuated bacteria in combination with one or more additional immunogenic components. Such a combination vaccine produces in the vaccinated animal a surprisingly greater effect than that expected by simply adding the effects of each component administered separately. Thus, a combination vaccine may stimulate a synergistic production of antibody in animals.

Other immunogenic components useful in the combination vaccines herein contemplated include, but are not limited to, canine distemper (CD) virus, canine adenovirus type 2 (CAV-2), canine parainfluenza (CPI) virus, canine parvovirus (CPV), canine coronavirus (CCV), canine herpesvirus, and rabies virus. Antigens from these immunogens for use in the vaccine compositions of the present invention can be in the form of a modified live viral preparation or an inactivated viral preparation. Methods of attenuating virulent strains of these viruses and methods of making an inactivated viral preparation are known in the art and are described in, e.g., U.S. Pat. Nos. 4,567,042 and 4,567,043.

In accordance with the present invention, the combination vaccines generally include a veterinary-acceptable carrier. A veterinary-acceptable carrier includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others.

One or more antigens from other pathogens, and the veterinary-acceptable carrier can be combined in any convenient and practical manner to form a combination vaccine composition, e.g., by admixture, solution, suspension, emulsification, encapsulation, absorption and the like, and can be made in formulations such as tablets, capsules, powder, syrup, suspensions that are suitable for injections, implantations, inhalations, ingestions or the like.

Vaccines of the present invention can be prepared by combination of at least one of the inactivated or attenuated bacteria with a pharmaceutically acceptable carrier and, preferably, an adjuvant.

Suitable preparations of the vaccines of the present invention include injectables, either liquid solutions or suspensions. Solid forms suitable for solution in, or suspension in, a liquid pharmaceutically acceptable carrier prior to injection may also be prepared. The vaccine preparation may be emulsified. The active immunogenic component, is preferably mixed with an adjuvant which is pharmaceutically acceptable and compatible with the active immunogenic component. Suitable adjuvants include, but are not limited to: mineral gels, e.g., aluminum hydroxide; surface active substances such as lysolecithin; glycosides, e.g., saponin derivatives such as Quil A or GPI-0100 (U.S. Pat. No. 5,977,081); cationic surfactants such as DDA, pluronic polyols; polyanions; non-ionic block polymers, e.g., Pluronic F-127 (B.A.S.F., USA); peptides; mineral oils, e.g. Montanide ISA-50 (Seppic, Paris, France), carbopol, Amphigen (Hydronics, Omaha, Nebr. USA), Alhydrogel (Superfos Biosector, Frederikssund, Denmark) oil emulsions, e.g. an emulsion of mineral oil such as BayolF/Arlacel A and water, or an emulsion of vegetable oil, water and an emulsifier such as lecithin; alum, cholesterol, rmLT, cytokines and combinations thereof. The immunogenic component may also be incorporated into liposomes, or conjugated to polysaccharides and/or other polymers for use in a vaccine formulation. Additional substances that can be included in a product for use in the present methods include, but are not limited to one or more preservatives such as disodium or tetrasodium salt of ethylenediaminetetracetic acid (EDTA), merthiolate, and the like.

The subject to which the vaccine is administered is preferably a companion animal, most preferably, a dog or cat.

It is preferred that the vaccine of the invention, when in a vaccine formulation, be present in unit dosage form. For purposes of this invention, an immunogenic amount, when administered comprises about 1×10⁴ to about 1×10¹³ inactivated bacterial cells. In a vaccine formulation containing multiple components, the same or lesser immunogenic amounts can usefully be employed.

Appropriate therapeutically effective doses can be determined readily by those of skill in the art based on the above immunogenic amounts, the condition being treated and the physiological characteristics of the animal. Accordingly, a vaccine preparation provides a dosage of a sterile preparation of an immunogenic amount of the active ingredient(s), where the active ingredient is at least one bacteria. In the presence of additional active agents, these unit dosages can be readily adjusted by those of skill in the art.

A desirable dosage regimen involves administration of at least one dose of desired vaccine composition, where the antigenic content of each fraction is as stated above. Effective doses (immunizing amounts) of the vaccines of the invention may also be extrapolated from dose-response curves derived from model test systems. The mode of administration of the vaccines of the invention can be any suitable route that delivers the vaccine to the host. These include but are not limited to oral, intradermal, intramuscular, intraperitoneal, subcutaneous, intranasal routes, and via scarification (scratching through the top layers of skin, e.g., using a bifurcated needle). However, the vaccine is preferably administered subcutaneously or by intramuscular injection. Other modes of administration can also be employed, where desired, such as intradermally, intravenously, intranasally, or intratonsillarly.

Studies have shown that, for each of the above described vaccine compositions, a primary immunization of young animals (after 8 weeks of age) is desirably initiated, with booster doses administered at 12 weeks and 16 weeks of age. Annual re-vaccination is recommended.

The vaccine of the present invention is administered and dosed in accordance with good medical practice, taking into account the clinical condition of the individual subject, the site and method of administration, scheduling of administration, subject age, sex, body weight and other factors known to medical practitioners.

The invention further provides kits for the prevention periodontal disease in companion animals. In one embodiment, the kit provides a container comprising a therapeutically effective amount of a composition, which prevents periodontal disease in companion animals. Also provided in the same or different container is a pharmaceutically acceptable carrier that may be used in the composition. The kit can additionally include an adjuvant that can be used to aid in creating the response to the composition of the present invention. Also, the kit can include a dispenser for dispensing the composition, preferably in unit dosage form. The dispenser can, for example, comprise metal or plastic foil, such as a blister pack. The kit can be accompanied by a label or printed instructions describing administration of the composition to prevent periodontal disease in a companion animal. Compositions comprising a vaccine composition of the present Invention formulated in a pharmaceutically acceptable carrier can also be prepared, placed in an appropriate container, and labeled for treatment of the indicated periodontal condition.

### Determination of Vaccine Efficacy

The specific mechanism of protection induced by the vaccines and vaccine compositions compositions of the present invention is the induction of the antibody and/or cellular immune response in vaccinated animals, as indicated by the in vivo animal tests described below.

The bacteria, vaccines, and vaccine compositions of the present invention are useful in treating or preventing companion animal periodontal disease, bovine foot rot, coronary heart disease (dogs), or systemic infections (dogs). The present invention is further illustrated by the following non-limiting example and accompanying tables.

### EXAMPLE 1

### Companion Animal Crevicular Fluid Sample

Microbial samples were taken from dogs and cats examined at veterinary clinics for periodontal treatment, or dogs examined at certain recognized facilities for normal check-ups. Dogs with periodontal pockets >3 mm and cats with periodontal pockets >2 mm were included in this study. Dental indices (gingival index and periodontal index) and the periodontal pocket depths were recorded. Individual coarse absorbent paper points (Henry Schein; Melville, N.Y.) were aseptically inserted into the periodontal pocket. Upon removal, the paper points were immediately inserted into vials containing Pre-Reduced Anaerobically Sterile (PRAS) Anaerobic Dental Transport (ADT) Medium (Anaerobe Systems; Morgan Hills, Calif.).

Vials were transferred into a Bactron IV anaerobic chamber (Sheldon Manufacturing, Cornelius, Oreg.) and processed under 90% N₂, 5% H₂, 5% CO₂. The paper points were aseptically placed into 50 μl of PRAS Brain Heart Infusion (BHI), PYG or SSYG media (Anaerobe Systems) and vortexed for 30 seconds. Dilutions of 1:100 and 1:1000 were prepared in BHI, PYG or SSYG media. Aliquots of 100 μl of the 1:100 and 1:1000 dilutions were spread on PRAS Burcella Blood Agar (BRU) plates (Anaerobe Systems). The plates were incubated at 37° C. in the anaerobic chamber for five to seven days. The total number of bacterial colonies and the number of Black Pigmented Anaerobic Bacteria (BPAB) colonies were counted. Individual BPAP colonies were transferred to new BRU plates and re-incubated as above.

### Clinical Isolate Characterization

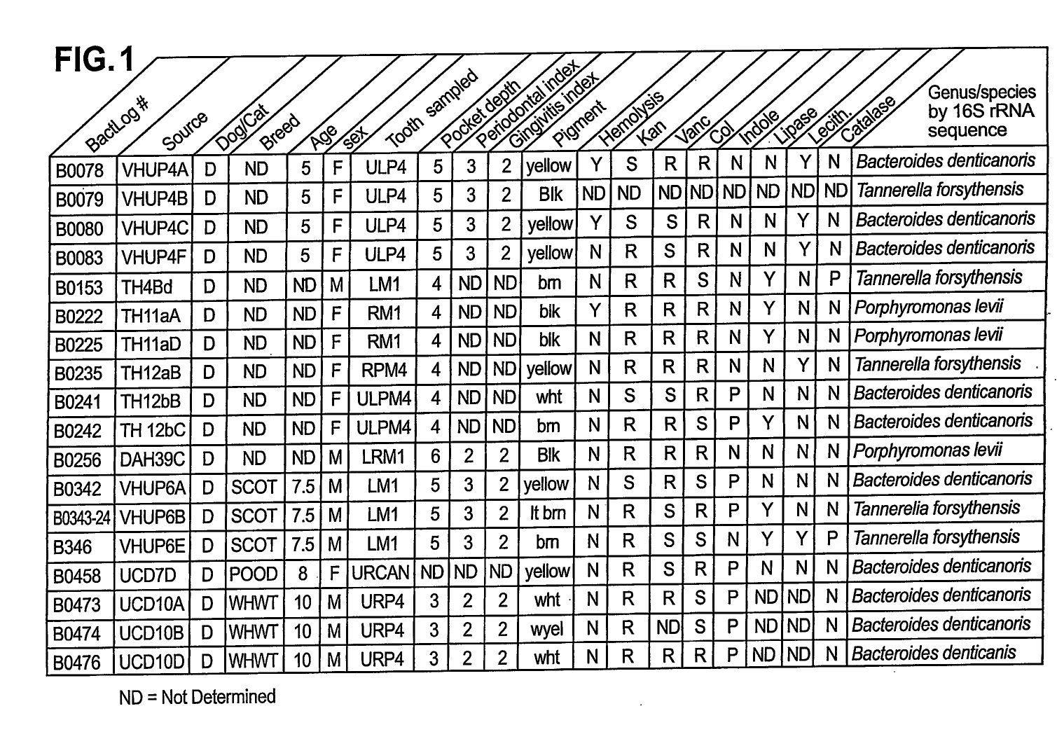

Each clinical isolate was subjected to a number of biochemical analyses and 16S rRNA DNA sequence analysis, using primers D0056 and D0057 (Seq. ID No. 1 and Seq. ID No. 2; Table 1), to determine genus and species. Individual isolates were streaked on BRU plates. Kanamycin, Vancomycin, and Colistin disks (Anaerobe Systems) were placed on the agar surface to determine the KVC resistance patterns of each isolate. Purified colonies were also subjected to the indole and catalase tests (Anaerobe Systems). Individual isolates were transferred to Egg Yolk Agar (EYA) plates (Anaerobe Systems) in order to determine lipase and lecithinase production patterns. This data is shown in FIG. 1 below.

Periodontal Index refers to a systematic classification of the severity of periodontal disease, taking into account multiple aspects of this multifaceted disease. These include, but are not limited to: pocket depth, attachment loss, bleeding on probing, dental mobility, and gingivitis. Gingival Index refers to a systematic classification of the severity of gingival inflammation. Signs observed which impact this classification include, but are not limited to: degree of edema, color, spontaneous bleeding, gingival recession, and hyperplasticity.

The partial 16S rRNA sequences from the five Tannerella forsythensis isolates characterized revealed 100% identity within the approximately 520-bp region. The three Porphyromonas levii isolates were greater than 99% identical within the partial 16S rRNA sequences analyzed, differing only at one nucleotide (position 13 in SEQ ID NO. 4).

### Identification of a Novel Species of Bacteroides (Bacteroides denticanoris)

During the course of this study we identified numerous clinical isolates whose 16S rRNA sequences did not have highly similar matches in the available databases, indicating that the bacteria may represent novel isolates. One group of these isolates (Table 2-below) appeared to represent a novel species. Based on the data presented herewithin, we propose that this group of bacterial isolates be called Bacteroides denticanoris sp. nov. The type strain of B. denticanoris is strain B78^{T} (=ATCC PTA-5881).

TABLE 2
Canine clinical bacterial isolates utilized in this study.

| | | | | SEQ ID |
|---|---|---|---|---|
| | | Periodontal | | NO. of 16S |
| | | pocket | | RNA |
| Strain | Tooth | depth (mm) | Location | Sequence |
| B denticanoris B78^{T} | Upper left pre molar #4 | 5 | Pennsylvania | 3 (fragment |
| | | | | and 6 (full |
| | | | | length) |
| B. denticanoris B80 | Upper left pre molar #4 | 5 | Pennsylvania | 7 |
| B. denticanoris B83 | Upper left pre molar #4 | 5 | Pennsylvania | 8 |
| B. denticanoris B241 | Upper left pre molar #4 | 4 | Indiana | 9 |
| B. denticanoris B242 | Upper left pre molar #4 | 4 | Indiana | 10 |
| B. denticanoris B342 | Lower left first molar | 5 | Pennsylvania | 11 |
| B. denticanoris B458 | Upper right canine | ND* | California | 12 |
| B. denticanoris B473 | Upper right pre molar #4 | 3 | California | 13 |
| B. denticanoris B474 | Upper right pre molar #4 | 3 | California | 14 |
| B. denticanoris B476 | Upper right pre molar #4 | 3 | California | 15 |

*ND, not determined

Phenotypic Characterization of Bacteroides denticanoris B78^{T}

B. denticanoris B78^{T} was isolated from a five-year old female mixed breed dog with periodontal disease. Clinically, the dog had a periodontal index score of 3, and a gingival index score of 2 (Harvey, 1998). A paper point sample was obtained from the upper left, fourth premolar, which had a periodontal pocket depth of 5 mm. The sample was process as described above. The purified cells were Gram-negative, non-spore forming, non-motile, rod shaped, and catalase-negative. Colonies formed after approximately five days of anaerobic incubation on Brucella blood agar at 37° C. Colonies of B. denticanoris B78^{T} began to pigment (tan to black) after 5-7 days of Incubation. The isolate appeared hemolytic on Brucella blood agar, sensitive to kanamycin, and resistant to both vancomycin and colistin (antibiotic discs from Anaerobe Systems). Colonies on egg yolk agar (Anaerobe Systems) demonstrated lecithinase activity, but not lipase activity. There was no evidence of bacterial swarming since distinct colonies appeared on numerous media types (data not shown).

Biochemical Analysis

B. denticanoris B78^{T} was subjected to biochemical analysis using the RapID ANA II clinical test kit (Remel; Lenexa, Kans.). Briefly, three cultures of B. denticanoris B78^{T} on Brucella blood agar were resuspended to McFarland # 3 equivalency. The suspensions were added to the test wells and incubated for 4 hours at 37° C. Following the incubation, results for the 18 different biochemical tests were recorded.

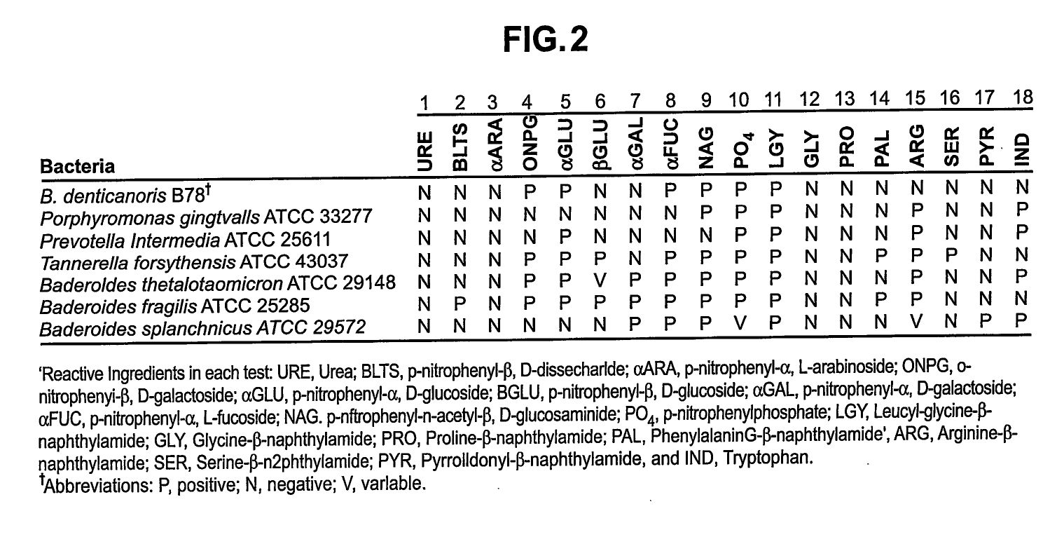

FIG. 2 shows the results of RapID ANA II testing for B. denticanoris B78^{T} as well as six control bacteria. Of the 18 tests performed by the RapID ANA II kit, six (ONPG, βGLU, αFUC, NAG, PO₄, and LGY) were positive for B. denticanoris B78^{T}. In comparison, Porphyromonas gingivalis ATCC 33277, Prevotella intermedia ATCC 25611, Tannerella forsythensis ATCC 43037, Bacteroides thetaiotaomicron ATCC 29148, Bacteroides fragilis ATCC 25285, and Bacteroides splanchnicus ATCC 29572 yielded 5, 4, 10, 12, 9, and 8 positive tests, respectively.

Phylogenetic Analysis

The full-length 16S rRNA genes from B. denticanoris B78^{T} was PCR amplified in triplicate using the primers D134 (5′-GAGTTTGATCCTGGCTCAGG-3′-SEQ ID NO:16) and D57 (5′-CCCGGGAACGTATTCACCG-3′-SEQ ID NO:17) (Invitrogen Corp.). Slots, J., et al. Clin Infect Dis 20 Suppl 2, S304-S307 (1995).

The PCR products were pooled, purified, desalted, and subjected to direct DNA sequence analysis. BLAST-N (Altschul, S. F. et al. J Mol Biol 215, 403-410) (1990). searches of the non-redundant nucleotide database at the National Center for Biotechnology Information using the B. denticanoris B78^{T} 16S rRNA gene sequence indicated that the B. denticanoris B78^{T} isolate was related to members of the Bacteroides genus. The most closely related sequence in the database was the 16S rRNA gene sequence of Bacteroidetes sp. 0103 800 (accession number AJ416906), showing 97% identity over 1,463 bp. Bacteroides sp. 0103-800 was isolated from an anaerobic brain abscess.

Phylogenetic analysis based on 16S rRNA gene sequences was performed using the CLUSTAL X version 1.81 software. Phylogenetic trees were generated using the neighbor-joining method (Saitou, N. & Nei, M. Mol Biol Evol 4, 406-425) (1987).

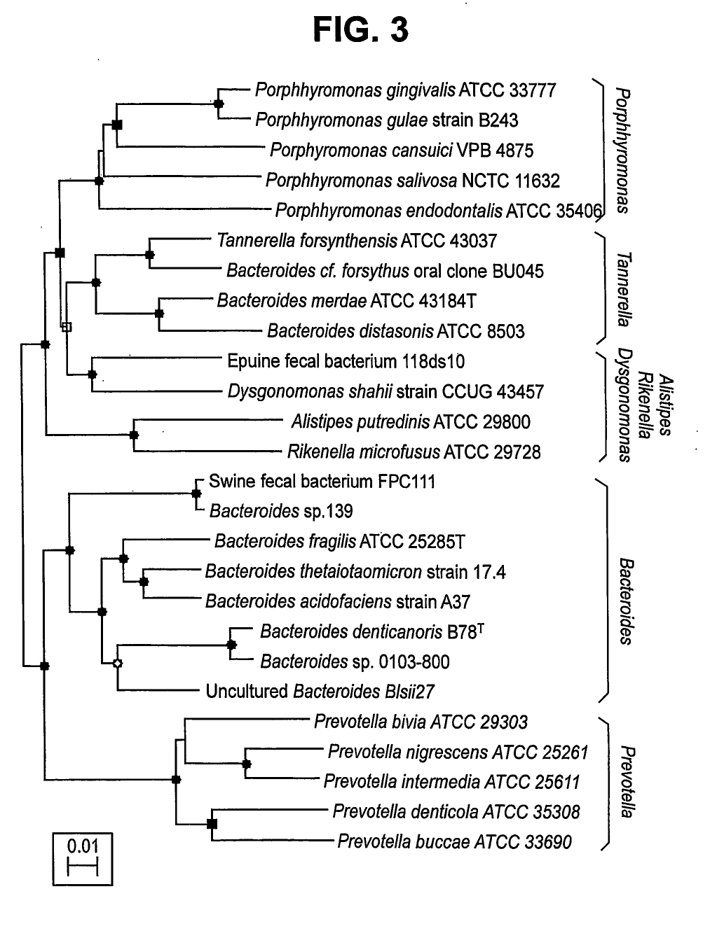

Bootstrap values were obtained using 1000 replicates. FIG. 3 shows the results of phylogenetic analysis for the B. denticanoris B78^{T} isolate. The placement of the major genera (Porphyromonas, Bacteroides, Prevotella, Tannerella, etc.) is in agreement with previously published phylogenetic trees for the cytophaga-flavobacter-bacteroides (CFB) group (Paster, et al (1994). J Bacteriol 176, 725-732., Shah, H. N., et al. Bacteroides, Prevotella, and Porphyromonas. In Microbiology and microbial infections, pp. 1305-1330. Edited by A. Balows and B. I. Duerden. London: Oxford University Press (1998).

B. denticanoris B78^{T}, Bacteroides sp. 0103-800, and the uncultured Bacteroidetes Bisii27 isolate are grouped In an off branch of the B. fragilis group that also contains B. acidofaciens and B. thetaiotaomicron (FIG. 1). A bootstrap confidence value of 99.9% on the branch point of the B. denticanoris B78^{T} sub-group from the B. fragilis sub-group adds strength to the phylogenetic placement of this newly identified organism.

An approximately 560-bp region of the 16S rRNA gene from nine other canine clinical isolates of B. denticanoris (Table 1) was PCR amplified (in triplicate) using the D56 and D57 primers described above. The PCR products were purified, desalted, and pooled. The DNA sequence of the PCR products was then determined. The results are detailed below in Table 3. The isolates were typed based on their 16S rRNA DNA sequence. Individual, well-isolated colonies were utilized as template for polymerase chain reactions (PCR) amplification of the 16S rRNA region using primers D0056 and D0057 (Seq. ID No. 1 and Seq. ID No. 2; Table 1) in triplicate. The PCR was carried out in 50 μl reaction volumes containing 1× PCR buffer (Life Technologies; Rockville, Md.), 1.0 mM MgCl₂, 1.25 μM each primer, 300 μM each deoxy-NTP, and 2.5 U Platinum Pfx DNA Polymerase (Life Technologies). The following PCR cycle conditions were utilized: a two minute denaturation step at 94° C.; 30 cycles of denaturation at 94° C. for 40 seconds, annealing at 60° C. for 40 seconds, and extension at 72° C. for one minute; a final extension step at 72° C. for two minutes; and a final cooling step to 4° C. A GeneAmp 9700 thermocycler (Perkin Elmer Applied Biosystems; Foster City, Calif.) was utilized for all POR amplifications.

The resulting PCR products were purified using the PCR preps kits (Promega Corp.; Madison, Wisc.) and pooled by isolate. The purified PCR products were then desalted by drop analysis against 25 ml sterile water using a 0.025 μm nitrocellulose filter (Millipore Corp.; Bedford, Mass.). The purified, desalted PCR products were subjected to DNA sequence analysis using the DyeDeoxy termination reaction on an ABI automated DNA sequencer. Synthetic oligonucleotide primers D0056 and D0057 (Seq. ID No. 1-2, respectively; 1) were used to obtain double stranded DNA sequence. The resulting DNA sequences were used to search publicly available DNA databases using a BLAST-N program publicly available from The National Center for Biotechnology Information, USA.

TABLE 3

DNA sequence identification listing. All oligonucleotide
primers were synthesized by Gibco-BRL (USA).

| | | |
|---|---|---|
| SEQ ID NO. | Name | DNA Sequence |

| | | |
|---|---|---|
| 7 | Bacteroides | CAGTAAACGATGAATACTCGCTGTTTGCGATACACTGTAAGCGGCCAAGCGAAA |
| | denticanoris | GCGTTAAGTATTCCACCTGGGGAGTACGCCGGCAACGGTGAAACTCAAAGAA |
| | (B80) 16S rRNA | TTGACGGGGGCCCGCACAAGCGGAGGAACATGTGGTTTAATTCGATGATACGC |
| | polynucleotide | GAGGAACCTTACCCGGGCTTAAATTGCGCTGGCTTTTACCGGAAACGGTATTTT |
| | sequence | CTTCGGACCAGCGTGAAGGTGCTGCATGGTTGTCGTCAGCTCGTGCCGTGAG |
| | | GTGTCGGCTTAAGTGCCATAACGAGCGCAACCCTTATCTTTAGTTACTAACAGT |
| | | TTTGCTGAGGACTCTAAAGAGACTGCCGTCGTAAGATGCGAGGAAGGTGGGGA |
| | | TGACGTCAAATCAGCACGGCCCTTACGTCCGGGGCTACACACGTGTTACAATG |
| | | GGGAGCACAGCAGGTTGCTACACGGCGACGTGATGCCAATCCGTAAAACTCCT |
| | | CTCAGTTCGGATCGAAGTCTGCAACCCGACTTCGTGAAGCTGGATTCGCTAGT |
| | | AATCGCGCATCAGCCACGGCGCGGTGAATAC |
| 8 | Bacteroides | CAGTAAACGATGAATACTCGCTGTTTGCGATACACTGTAAGCGGCCAAGCGAAA |
| | denticanoris | GCGTTAAGTATTCCACCTGGGGAGTACGCCGGCAACGGTGAAACTCAAAGGAA |
| | (B83) 16S rRNA | TTGACGGGGGCCCGCACAAGCGGAGGAACATGTGGTTTAATTCGATGATACGC |
| | polynucleotide | GAGGAACCTTACCCGGGCTTAAATTGCGCTGGCTTTTACCGGAAACGGTATTTT |
| | sequence | CTTCGGACCAGCGTGAAGGTGCTGCATGGTTGTCGTCAGCTCGTGCCGTGAG |
| | | GTGTCGGCTTAAGTGCCATAACGAGCGCAACCCTTATCTTTAGTTACTAACAGT |
| | | TTTGCTGAGGACTCTAAAGAGACTGCCGTCGTAAGATGCGAGGAAGGTGGGGA |
| | | TGACGTCAAATCAGCACGGCCCTTACGTCCGGGGCTACACACGTGTTACAATG |
| | | GGGAGCACAGCAGGTTGCTACACGGCGACGTGATGCCAATCCGTAAAACTCCT |
| | | CTCAGTTCGGATCGAAGTCTGCAACCCGACTTCGTGAAGCTGGATTCGCTAGT |
| | | AATCGCGCATCAGCCACGGCGCGGTGAATAC |
| 9 | Bacteroides | GCACAGTAAACGATGAATACTCGCTGTTTGCGATACACTGTAAGCGGCCAAGC |
| | denticanoris | GAAAGCGTTAAGTATTCCACCTGGGGAGTACGCCGGCAACGGTGAAACTCAAA |
| | (B241) 16S rRNA | GGAATTGACGGGGGCCCGCACAAGCGGAGGAACATGTGGTTTAATTCGATGAT |
| | polynucleotide | ACGCGAGGAACCTTACCCGGGCTTAAATTGCGCTGGCTTTTACCGGAAACGGT |
| | sequence | ATTTTCTTCGGACCAGCGTGAAGGTGCTGCATGGTTGTCGTCAGCTCGTGCCG |
| | | TGAGGTGTCGGCTTAAGTGCCATAACGAGCGCAACCCTTATCTTTAGTTACTAA |
| | | CAGTTTTGCTGAGGACTCTAAAGAGACTGCCGTCGTAAGATGCGAGGAAGGTG |
| | | GGGATGACGTCAAATCAGCACGGCCCTTACGTCCGGGGCTACACACGTGTTAC |
| | | AATGGGGAGCACAGCAGGTTGCTACACGGCGACGTGATGCCAATCCGTAAAAC |
| | | TCCTCTCAGTTCGGATCGAAGTCTGCAACCCGACTTCGTGAAGCTGGATTCGCT |
| | | AGTAATCGCGCATCAACCACGGCGCGGTGAATA |
| 10 | Bacteroides | ACAGTAAACGATGAAATACTCGCTGTTTGCGATACACTGTAAGCGGCC |
| | denticanoris | AAGCGAAAGCGTTAAGTATTCCACCTGGGGAGTACGCCGGCAACGGTGAAACT |
| | (B242) 16S rRNA | CAAAGGAATTGACGGGGGCCCGCACAAGCGGAGGAACATGTGGTTTAATTCGA |
| | polynucleotide | TGATACGCGAGGAACCTTACCCGGGCTTAAATTGCGCTGGCTTTTACCGGAAA |
| | sequence | CGGTATTTTCTTCGGACCAGCGTGAAGGTGCTGCATGGTTGTCGTCAGCTCGT |
| | | GCCGTGAGGTGTCGGCTTAAGTGCCATAACGAGCGCAACCCTTATCTTTAGTTA |
| | | CTAACAGTTTTGCTGAGGACTCTAAAGAGACTGCCGTCGTAAGATGCGAGGAA |
| | | GGTGGGGATGACGTCAAATCAGCACGGCCCTTACGTCCGGGGCTACACACGT |
| | | GTTACAATGGGGAGCACAGCAGGTTGCTACACGGCGACGTGATGCCAATCCGT |
| | | AAAACTCCTCTCAGTTCGGATCGAAGTCTGCAACCCGACTTCGTGAAGCTGGAT |
| | | TCGCTAGTAATCGCGCATCAACCACGGCGCGGTGAATA |
| 11 | Bacteroides | CGCACAGTAAACGATGAATACTCGCTGTTTGCGATACACTGTAAGCGGCCAAG |
| | denticanoris | CGAAAGCGTTAAGTATTCCACCTGGGGAGTACGCCGGCAACGGTGAAACTCAA |
| | (B342) 16S rRNA | AGGAATTGACGGGGGCCCGCACAAGCGGAGGAACATGTGGTTTATTCGATGA |
| | polynucleotide | TACGCGAGGAACCTTACCCGGGCTTAAATTGCGCTGGCTTTTACCGGAAACGG |
| | sequence | TATTTTCTTCGGACCAGCGTGAAGGTGCTGCATGGTTGTCGTCAGCTCGTGCC |
| | | GTGAGGTGTCGGCTTAAGTGCCATAACGAGCGCAACCCTTATCTTTAGTTACTA |
| | | ACAGTTTTGCTGAGGACTCTAAAGAGACTGCCGTCGTAAGATGCGAGGAAGGT |
| | | GGGGATGACGTCAAATCAGCACGGCCCTTACGTCCGGGGCTACACACGTGTTA |
| | | CAATGGGGAGCACAGCAGGTTGCTACACGGCGACGTGATGCCAATCCGTAAAA |
| | | CTCCTCTCAGTTCGGATCGAAGTCTGCAACCCGACTTCGTGAAGCTGGATTCG |
| | | CTAGTAATCGCGCATNACCACGGNGCGGTGAATAC |
| 12 | Bacteroides | GCACAGTAAACGATGAATACTCGCTGTTTGCGATACACTGTAAGCGGCCAAGC |
| | denticanoris | GAAAGCGTTAAGTATTCCACCTGGGGAGTACGCCGGCAACGGTGAAACTCAAA |
| | (B458) 16S rRNA | GGAATTGACGGGGGCCCGCACAAGCGGAGGAACATGTGGTTTAATTCGATGAT |
| | polynucleotide | ACGCGAGGAACCTTACCCGGGCTTAAATTGCGCTGGCTTTTACCGGAAACGGT |
| | sequence | ATTTTCTTCGGACCAGCGTGAAGGTGCTGCATGGTTGTCGTCAGCTCGTGCCG |
| | | TGAGGTGTCGGCTTAAGTGCCATAACGAGCGCAACCCTTATCTTTAGTTACTAA |
| | | CAGTTTTGCTGAGGACTCTAAAGAGACTGCCGTCGTAAGATGCGAGGAAGGTG |
| | | GGGATGACGTCAAATCAGCACGGCCCTTACGTCCGGGGCTACACACGTGTTAC |
| | | AATGGGGAGCACAGCAGGTTGCTACACGGCGACGTGATGCCAATCCGTAAAAC |
| | | TCCTCTCAGTTCGGATCGAAGTCTGCAACCCGACTTCGTGAAGCTGGATTCGCT |
| | | AGTAATCGCGCATCAGCCACGGCGCGGTGAATA |
| 13 | Bacteroides | CACAGTAAACGATGAATACTCGCTGTTTGCGATACACGGTAAGCGGCCAAGCG |
| | denticanoris | AAAGCGTTAAGTATTCCACCTGGGGAGTACGCCGGCAACGGTGAAACTCAAAG |
| | (B473) 16S rRNA | GAATTGACGGGGGCCCGAACAAGCGGAGGAACATGTGGTTTAATTCGATGATA |
| | polynucleotide | CGCGAGGAACCTTACCCGGGCTTAAATTGCGCTGGCTTTTACCGGAAACGGTA |
| | sequence | TTTTCTTCGGACCAGCGTGAAGGTGCTGCATGGTTGTCGTCAGCTCGTGCCGT |
| | | GAGGTGTCGGCTTAAGTGCCATAACGAGCGCAACCCTTATCTTTAGTTACTAAC |
| | | AGTTTTGCTGAGGACTCTAAAGAGACTGCCGTCGTAAGATGCGAGGAAGGTGG |
| | | GGATGACGTCAAATCAGCACGGCCCTTACGTCCGGGGCTACACACGTGTTACA |
| | | ATGGGGAGCACAGCAGGTTGCTACACGGCGACGTGATGCCAATCCGTAAAACT |
| | | CCTCTCAGTTCGGATCGAAGTCTGCAACCCGACTTCGTGAAGCTGGATTCGCT |
| | | AGTAATCGCGCATCAGCCACGGCGCGGTGAATAC |
| 14 | Bacteroides | ACAGTAAACGATGAATACTCGCTGTTTGCGATACACGGTAAGCGGCCAAGCGA |
| | denticanoris | AAGCGTTAAGTATTCCACCTGGGGAGTACGCCGGCAACGGTGAAACTCAAAGG |
| | (B474) 16S rRNA | AATTGACGGGGGCCCGCACAAGCGGAGGAACATGTGGTTTAATTCGATGATAC |
| | polynucleotide | GCGAGGAACCTTACCCGGGCTTAAATTGCGCTGGCTTTTACCGGAAACGGTAT |
| | sequence | TTTCTTCGGACCAGCGTGAAGGTGCTGCATGGTTGTCGTCAGCTCGTGCCGTG |
| | | AGGTGTCGGCTTAAGTGCCATAACGAGCGCAACCCTTATCTTTAGTTACTAACA |
| | | GTTTTGCTGAGGACTCTAAAGAGACTGCCGTCGTAAAGATGCGAGGAAGGTGGG |
| | | GATGACGTCAAATCAGCACGGCCCTTACGTCCGGGGCTACACACGTGTTACAA |
| | | TGGGGAGCACAGCAGGTTGCTACACGGCGACGTGATGCCAATCCGTAAAACTC |
| | | CTCTCAGTTCGGATCGAAGTCTGCAACCCGACTTCGTGAAGCTGGATTCGCTA |
| | | GTAATCGCGCATCAGCCACGGCGCGGTGAATAC |
| 15 | Bacteroides | CACAGTAAACGATGAATACTCGCTGTTTGCGATACACGGTAAGCGGCC |
| | denticanoris | AAGCGAAAGCGTTAAGTATTCCACCTGGGGAGTACGCCGGCAACGGTGAAACT |
| | (B476) 16S rRNA | CAAAGGAATTGACGGGGGCCCGCACAAGCGGAGGAACATGTGGTTTAATTCGA |
| | polynucleotide | TGATACGCGAGGAACCTTACCCGGGCTTAAATTGCGCTGGCTTTTACCGGAAA |
| | sequence | CGGTATTTTCTTCGGACCAGCGTGAAGGTGCTGCATGGTTGTCGTCAGCTCGT |
| | | GCCGTGAGGTGTCGGCTTAAGTGCCATAACGAGCGCAACCCTTATCTTTAGTTA |
| | | CTAACAGTTTTGCTGAGGACTCTAAAGAGACTGCCGTCGTAAGATGCGAGGAA |
| | | GGTGGGGATGACGTCAAATCAGCACGGCCCTTACGTCCGGGGCTACACACGT |
| | | GTTACAATGGGGAGCACAGCAGGTTGCTACACGGCGACGTGATGCCAATCCGT |
| | | AAAACTCCTCTCAGTTCGGATCGAAGTCTGCAACCCGACTTCGTGAAGCTGGAT |
| | | TCGCTAGTAATCGCGCATCAGCCACGGCGCGGTGAATAC |

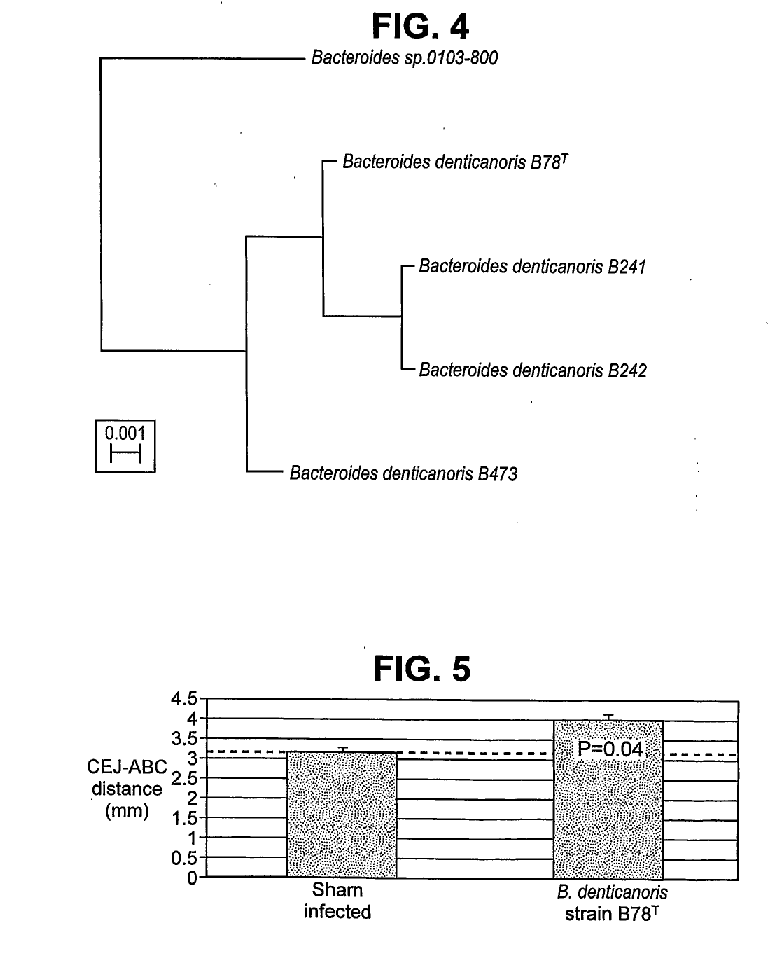

The partial 16S rRNA sequences from the ten B. denticanoris isolates were found to cluster into four sequences groups (B78^{T}, B80, B83, B342, and B458; B241; B242; and B473, B474, and B476). All isolates within a group had identical 16S rRNA sequences in this approximately 560-bp region. FIG. 4 shows the results of phylogenetic analysis of the B. denticanoris isolates partial 16S rRNA gene sequences. Between all of the B. denticanoris isolates, there is a 99.5% DNA sequence identity within the 560-bp region. Based on this observation, we conclude that all of these isolates are varying strains of the same species. Additionally, strains of this species were found in geographically distant locations (Pennsylvania and California).

A complete listing of all the isolates and their respective characteristics is located in FIG. 1.

The distribution of isolates is shown in Table 4.

TABLE 4
Summary of the number of dogs identified to harbor indicated bacterial
species.

| | # dog | # | % positive |
|---|---|---|---|
| Isolate | isolates | dogs | dogs |
| Bacteroides denticanoris | 10 | 5 | 10 |
| Porphyromonas levii | 3 | 2 | 4 |
| Tannerella forsythensis | 5 | 4 | 8 |

The table above indicates the number of isolates, as well as the number and percentage of dogs from which the indicated bacterial species were isolated.

The following companion animal periodontal isolates were deposited with the American Type Culture Collection (ATCC), 10801 University Blvd., Manassas, Va., 20110, USA, Bacteroides denticanoris (B78;(PTA-5881), Porphyromonas levii (B222; (PTA-5882), and Tannerella forsythensis (B343-24; PTA-6063).

### Culture Conditions for Bacterial Species

Since the standard growth media for many anaerobic bacteria (Brain Heart Infusion [BHI] and Chopped Meat Carbohydrate [CMC] media) contain animal product, which are not amenable for vaccine production, a growth medium that does not contain these ingredients was sought. Various media compositions, with and without the addition of hemin and vitamin K, were tested for their ability to support growth equivalent to that of growth of BHI or CMC. Both the PYG-complete and the SSYG media supported the growth of Bacteroides denticanis, P. levii, and T. forsythensis. The PYG-complete or SSYG-complete media were chosen as the growth media due to their ability to yield high-density cultures during fermentation. The PYG medium contains the following ingredients: 3% phytone (Becton Dickinson; Cockeysville, Md.), 0.3% yeast extract (Becton Dickinson), 0.3% glucose (Sigma Corp.; St. Louis, Mo.), 0.05% sodium thioglycollate (Becton Dickinson), 0.5% sodium chloride (Sigma Corp.), 5 μg/ml hemin (Sigma Corp.) (added after autoclaving), 0.5 μg/ml menadione (Sigma Corp.) (added after autoclaving), and 0.2% sodium bicarbonate (Sigma Corp.), pH 7.0. Bacteroides denticanis, P. levii, and T. forsythensis. were routinely cultivated on Brucella blood agar plates (Anaerobe Systems) or in complete PYG medium or BHI at 37° C. in a Bactron IV anaerobic chamber (Shel Labs; Cornelius, Oreg.) under 90% N₂, 5% CO₂ for three to five days (plates) or 24 to 48 hours (liquid cultures). The SSYG medium contains the following ingredients: 5% soytone (Becton Dickinson), 0.3% yeast extract (Becton Dickinson), 0.3% glucose (EM Industries), 0.05% sodium thioglycollate (Becton Dickinson), 0.5% sodium chloride (Sigma Corp.), 0.2% sodium bicarbonate (Fisher), dH2O, pH 7.0 and hemin-menadione solution containing hemin solution of 5 μg/ml hemin, 1N sodium hydroxide in dH2O and menadione solution of 0.5 μg/ml in 95% ethanol.

### Pathogenicity Testing of Clinical Isolates

Bacteroides denticanoris and P. levii were tested for their pathogenicity in the mouse periodontal bone loss model. Three-week-old, age-matched male Balb/c CyJ mice (Jackson Laboratories; Bar Harbor, Me.) with estimated weights of 14-15 grams were utilized for this study. The animals were housed in positive pressure, barrier cage units. Food pellets, standard for the species, and water were provided ad libitum throughout the experiment. The bedding utilized was granular Bed O'Cobbs to minimize impaction in the gingival tissues. Following receipt, all animals were acclimatized for five to seven days. To reduce competing oral flora, animals were placed on a mixture of sulfamathoxazole and trimethoprim (10 ml drinking water; approximately 2 mg and 0.4 mg/ml, respectively) for ten days followed by a five-day washout period. Serum samples were taken from each mouse tail vein bleed. The animals were infected with 0.5 ml suspension of approximately 1×10¹⁰ cfu/ml of the appropriate bacterial strain in 1% carboxymethylcellulose by gavage. Additional drops were placed in the oral cavity. This infection was repeated two more times for a total of three times (Monday, Wednesday, and Friday).

Day 1 of the experiment was defined as the Tuesday following the first infection. All animals were sacrificed on Day 2. Post-infection serum was collected, as were microbial samples. The jaws of each mouse were defleshed, stained, and scored for horizontal bone loss microscopically. The scoring was repeated three times to reduce operator error. The average bone loss is expressed as the average bone loss/site/jaw in mm. Statistical analysis of the resulting data was done with Systat (version 9), SigmaStat (version 2), and SigmaPlot (version 2000) available from SPSS Science Inc. (Chicago, Ill.). Table 5 shows the numerical results for these isolates.

TABLE 5
Summary of the mouse periodontal disease pathogenicity trial.

| | | Mean Bone | Standard | |
|---|---|---|---|---|
| Isolate | Mice/group | Loss (mm) | Deviation | SEM |
| Sham | 16 | 3.35 | 0.473 | 0.383 |
| Bacteroides | 16 | 3.86 | 0.605 | 0.486 |
| denticanoris | | | | |
| P. levii | 16 | 3.53 | 0.460 | 0.371 |
| T. forsythensis | ND | ND | ND | ND |

ND = Not Done

These data indicate that the Bacteroides denticanoris isolate is capable of causing bone loss in the mouse model of periodontal disease. Although minimal, the Porphyromonas levii isolate did cause bone loss in the mouse periodontal model. The results for Bacteroides denticanoris are displayed graphically in FIG. 5.

Throughout this application, various patent and scientific publications, including United States patents, are referenced by author and year and patents by number. The disclosures of these publications and patents are hereby incorporated by reference in their entireties into this application in order to more fully describe the state of the art to which this invention pertains.

1. At least one isolated pigmented anaerobic bacterium comprising a 16S rRNA DNA sequence at least 95% homologous to a sequence selected from the group consisting of SEQ ID NOS: 3, 4, 5, 6, 9, 10 and 13 wherein the bacterium causes, either directly or in combination with other pathogenic agents periodontal disease in companion animals.

2. The bacterium according to claim 1 comprising a 16S rRNA DNA sequence at least 99% homologous to a sequence selected from the group consisting of SEQ ID NOS: 3, 4, 5, 6, 9, 10 and 13.

3. The bacterium according to claim 1 comprising a 16S rRNA DNA sequence at least 99.5% homologous to a sequence selected from the group consisting of SEQ ID NOS: 3, 4, 5, 6, 9, 10 and 13.

4. The bacterium according to claim 1 comprising a 16S rRNA DNA sequence selected from the group consisting of SEQ ID NOS: 3, 4, 5, 6, 9, 10 and 13.

5. The bacterium according to claim 1 which is Bacteroides denticanoris.

6. The bacterium according to claim 5 which is ATCC PTA-5881 or a bacterium having all of the identifying characteristics of ATCC PTA-5881.

7. The bacterium according to claim 1 which is Porphyromonas levii.

8. The bacterium according to claim 7 which is ATCC PTA-5882 or a bacterium having all of the identifying characteristics of ATCC PTA-5882

9. The bacterium according to claim 1 which is Tannerella forsythensis

10. The bacterium according to claim 9 which is ATCC PTA-6063 or a bacterium having all of the identifying characteristics of ATCC PTA-6063

11. The bacterium according to claim 1 wherein the companion animal is a cat or a dog.

12. An immunogenic composition comprising the pigmented anaerobic bacterium according to claim 1.

13. The immunogenic composition of claim 12 wherein the pigmented anaerobic bacterium is inactivated.

14. The immunogenic composition of claim 12 further comprising a pharmaceutically acceptable carrier.

15. A vaccine for treating or preventing periodontal disease in companion animals comprising an immunologically effective amount of the bacterium according to claim 1 and a pharmaceutically acceptable carrier.

16. The vaccine of claim 15 wherein the bacterium is inactivated.

17. The vaccine composition as in claim 15, further comprising an adjuvant.

18. A method for treating or preventing periodontal disease in companion animals comprising administering to a companion animal in need thereof, a vaccine composition according to claim 15.

19. A method for diagnosing periodontal disease in companion animals by analyzing a sample from the oral cavity of the companion animal wherein the presence of one or more pigmented anaerobic bacteria according to claim 1 in the sample is indicative of disease.

20. The method according to claim 19 wherein the presence of a polynucleotide comprising a 16S rRNA DNA sequence at least about 95% homologous to a sequence selected from the group consisting of SEQ ID NOS: 3, 4, 5, 6, 9, 10 and 13 in the sample is indicative of disease.

21. The method according to claim 20 wherein the presence of a polynucleotide comprising a 16S rRNA DNA sequence at least about 99% homologous to a sequence selected from the group consisting of SEQ ID NOS: 3, 4, 5, 6, 9, 10 and 13 in the sample is indicative of disease.

22. The method according to claim 20 wherein the presence of a polynucleotide comprising a 16S rRNA DNA sequence at least about 99.5% homologous to a sequence selected from the group consisting of SEQ ID NOS: 3, 4, 5, 6, 9, 10 and 13 in the sample is indicative of disease.

23. The method according to claim 20 wherein the presence of a polynucleotide comprising a 16S rRNA DNA sequence selected from the group consisting of SEQ ID NOS: 3, 4, 5, 6, 9, 10 and 13 in the sample is indicative of disease.

24. The method according to claim 19, wherein said analyzing step includes analyzing the sample using a method selected from the group consisting of PCR, hybridization, and antibody detection.

25. A kit comprising, in at least one container, a composition for treating and preventing periodontal disease in companion animals comprising an effective amount of at least one live or inactivated isolated pigmented anaerobic bacteria, of any of claims 1 through 11 and a pharmaceutically acceptable carrier; wherein the kit further comprises a set of printed instructions indicating that the kit is useful for treating or preventing periodontal disease in companion animals.

26. A kit according to claim 25, wherein said kit further comprises a means for dispensing said composition.

27. A kit comprising in at least one container an isolated DNA molecule comprising a nucleotide sequence of at least about 15 contiguous nucleotides selected from any of SEQ ID NOS: 3, 4, 5, 6, 9, 10 and 13, which hybridizes under highly stringent conditions to the complement of any of the nucleotide sequences depicted in SEQ ID NOS: 3, 4, 5, 6, 9, 10 and 13, and a second isolated DNA molecule comprising in a second container an isolated DNA molecule comprising a nucleotide sequence of at least about 15 contiguous nucleotides selected from the complement of any of the nucleotide sequences depicted in SEQ ID NOS: 3, 4, 5, 6, 9, 10 and 13 which hybridizes under highly stringent conditions to any of the nucleotide sequences depicted in SEQ ID NOS: 3, 4, 5, 6, 9, 10 and 13, wherein the kit further comprises a set of instructions indicating that the kit is useful for the detection of Bacteroides, Porphyromonas, or Tannerella spp.

28. A hybridization kit comprising in at least one container an isolated DNA molecule comprising a nucleotide sequence of at least about 15 contiguous nucleotides selected from any of SEQ ID NOS: 3, 4, 5, 6, 9, 10 and 13, or its complement, wherein the hybridization is specific to Bacteroides, Porphyromonas, or Tannerella spp. and wherein the kit further comprises a set of instructions indicating that the kit is useful for the detection of Bacteroides, Porphyromonas, or Tannerella spp.

29. The kit according to claim 28 wherein the hybridization is performed under highly stringent conditions.

30. A biologically pure culture of bacteria wherein the bacteria comprise a 16S rRNA DNA sequence at least about 99% homologous to a sequence selected from the group consisting of SEQ ID NOS: 3, 6, 9, 10 and 13.

31. The biologically pure culture of bacteria according to claim 30 wherein the 16S rRNA DNA sequence is at least about 99.5% homologous to a sequence selected from the group consisting of SEQ ID NOS: 3, 6, 9, 10 and 13.

32. The biologically pure culture of bacteria according to claim 30 wherein the 16S rRNA DNA sequence is selected from the group consisting of SEQ ID NOS: 3, 6, 9, 10 and 13.

33. The biologically pure culture of bacteria according to claim 30, wherein the biologically pure culture of bacteria is independently selected from: ATCC PTA-5881 or a culture having all of the identifying characteristics of ATCC PTA-5881; ATCC PTA-5882 or a culture having all of the identifying characteristics of ATCC PTA-5882: or ATCC PTA-6063 or a culture having all of the identifying characteristics of ATCC PTA-6063.

34. A biologically pure culture of bacteria which is ATCC PTA-5882 or a culture having all of the identifying characteristics of ATCC PTA-5882.

35. A biologically pure culture of bacteria which is ATCC PTA-6063 or a culture having all of the identifying characteristics of ATCC PTA-6063
